(19) United States
(12) Patent Application Publication
(10) Pub. No.: US 2023/0197190 A1
(43) Pub. Date: Jun. 22, 2023

(54) CONTROL METHOD, CONTROL DEVICE, NON-TRANSITORY COMPUTER-READABLE RECORDING MEDIUM, AND APPARATUS

(71) Applicant: Panasonic Intellectual Property Corporation of America, Torrance, CA (US)

(72) Inventors: Kotaro SAKATA, Osaka (JP); Tetsuji FUCHIKAMI, Osaka (JP)

(21) Appl. No.: 18/113,166

(22) Filed: Feb. 23, 2023

Related U.S. Application Data
(63) Continuation of application No. PCT/JP2021/023976, filed on Jun. 24, 2021.

(30) Foreign Application Priority Data
Sep. 2, 2020 (JP) 2020-147553

(51) Int. Cl. G16B 20/00; G16B 40/20

(52) U.S. Cl. CPC G16B 20/00; G16B 40/20

(57) ABSTRACT

A control method includes: acquiring identification information for specifying a user and gene information of the user associated with the identification information; determining a physical constitution of the user on the basis of the acquired gene information; deciding an apparatus control program for controlling the apparatus on the basis of the determined physical constitution; and transmitting the acquired identification information and control information for controlling execution of an apparatus of the decided one apparatus control program in association with each other.

### TECHNICAL FIELD

The present disclosure relates to a technique for controlling an apparatus.

### BACKGROUND ART

In recent years, studies have been conducted to cause apparatuses to execute control suitable for an individual user. For example, Patent Literature 1 discloses a technique of collecting information regarding a condition of a user before the user enters a building, and controlling equipment in the building on the basis of the collected information, thereby improving comfort of the user.

However, since in the technique of Patent Literature 1, the apparatus is not controlled in consideration of a physical constitution of an individual user, further improvement is required in order to cause the apparatus to execute control suitable for an individual user.

### CITATION LIST

### Patent Literature

Patent Literature 1: JP 2012-138820 A

### SUMMARY OF INVENTION

An object of the present disclosure is to provide a technique enabling an apparatus to execute control suitable for an individual user.

A control method according to one aspect of the present disclosure is a control method of an apparatus, the method comprising: by a computer acquiring identification information for specifying a user and gene information of the user associated with the identification information, determining a physical constitution of the user on the basis of the acquired gene information, deciding an apparatus control program for controlling the apparatus on the basis of the determined physical constitution, and transmitting, in association with each other, the acquired identification information and control information for causing the apparatus to execute the decided apparatus control program.

According to the present disclosure, it is possible to cause an apparatus to execute control suitable for an individual user.

### BRIEF DESCRIPTION OF DRAWINGS

FIG. 1 is a diagram illustrating an example of an overall configuration of a control system according to a first embodiment of the present disclosure.

FIG. 2 is a block diagram illustrating an example of a configuration of a cell collection device.

FIG. 3 is a block diagram illustrating an example of a configuration of a server.

FIG. 4 is a block diagram illustrating an example of a configuration of an apparatus.

FIG. 5 is an explanatory diagram of SNP.

FIG. 6 is an explanatory diagram of a type of SNP.

FIG. 7 is a flowchart illustrating an example of processing executed when the control system according to the first embodiment of the present disclosure decides an apparatus control program.

FIG. 8 is a flowchart illustrating an example of processing executed when the apparatus executes the apparatus control program.

FIG. 9 is a block diagram illustrating an example of a configuration of a server according to a second embodiment of the present disclosure.

FIG. 10 is a flowchart illustrating an example of processing of the server according to the second embodiment.

FIG. 11 is a flowchart illustrating an example of processing of a control system according to a third embodiment of the present disclosure.

FIG. 12 is a flowchart illustrating an example of processing executed when an apparatus executes an apparatus control program in the third embodiment of the present disclosure.

### DESCRIPTION OF EMBODIMENTS

(How One Aspect of the Present Disclosure Has Come about)

In recent years, speed-up and cost reduction of techniques for analyzing human genes are under way. Accordingly, a user can easily take a genetic testing at home or the like. The genetic testing is a test for examining a base sequence of DNA including A (adenine), T (thymine), C (cytosine), and G (guanine). The base sequence of DNA varies from a person to a person, and this difference brings about diversity of human physical constitutions. Therefore, it is possible to determine a physical constitution of a person by examining a difference in a base sequence of DNA. Then, in consideration of the determined physical constitution, it is possible to cause an apparatus to execute control suitable for an individual user.

However, it is not a conventional practice to determine a user's physical constitution from gene information such as a base sequence of DNA and execute control of an apparatus on the basis of the determined physical constitution. For example, although in Patent Literature 1 described above, at least one of information regarding a behavior history of a user and information regarding an environment in which the user was present before entering a building is used in estimating a condition of the user, a physical constitution of the user is not taken into consideration. Therefore, conventional techniques are insufficient to cause an apparatus to execute control suitable for an individual user.

Therefore, the present inventor has acquired knowledge that it is possible to accurately determine a physical constitution of an individual user by analyzing gene information of the user, thereby enabling an apparatus to execute control suitable for an individual user, and has arrived at each aspect of the present disclosure.

A control method according to one aspect of the present disclosure is a control method of an apparatus, the method comprising: by aa computer acquiring identification information for specifying a user and gene information of the user associated with the identification information, determining a physical constitution of the user on the basis of the acquired gene information, deciding an apparatus control program for controlling the apparatus on the basis of the determined physical constitution, and transmitting, in association with each other, the acquired identification information and control information for causing the apparatus to execute the decided apparatus control program.

According to this configuration, since a physical constitution of a user is determined on the basis of the gene information, the physical constitution of the user can be accurately determined. Then, an apparatus control program is decided the basis of the determined physical constitution, and the control information that causes the apparatus to execute the decided apparatus control program and the identification information are transmitted. Therefore, the apparatus can specify an apparatus control program corresponding to the identification information. As a result, the apparatus can execute control suitable for an individual user.

In the control method, the gene information may be information indicating a base sequence of a nucleic acid of the user.

According to this configuration, since the gene information is information indicating a base sequence of a nucleic acid of the user, a physical constitution of the user can be accurately determined.

The control method may further include detecting an expression of a predetermined gene on the basis of a temporal change in the acquired gene information, and deciding the apparatus control program on the basis of a detection result.

When a predetermined gene is expressed, a physical constitution of a user changes. According to the present configuration, since the expression of the predetermined gene is detected and the apparatus control program is decided on the basis of the detection result, it is possible to decide an appropriate apparatus control program in consideration of a change in a physical constitution of a user.

In the control method, the physical constitution may be at least one of thermal diathesis, taste sensitivity, and fatigue diathesis.

According to the present configuration, it is possible to control an apparatus suitable for a user in consideration of at least one of thermal diathesis, taste sensitivity, and fatigue diathesis.

In the control method, the control information may include the apparatus control program.

According to this configuration, since the apparatus control program is transmitted, it is possible to cause the apparatus to execute the apparatus control program without causing the apparatus to hold the apparatus control program in advance.

In the control method, the control information may include information for causing the apparatus having a plurality of apparatus control programs to execute the decided apparatus control program.

According to this configuration, it is possible to cause the apparatus to execute the apparatus control program without transmitting the apparatus control program.

In the control method, the physical constitution may be thermal diathesis, the apparatus may be an air conditioning apparatus, and the decision may include deciding an apparatus control program for the air conditioning apparatus according to the thermal diathesis of the user.

According to this configuration, it is possible to cause the apparatus to execute appropriate control according to the thermal diathesis of the user.

In the above control method, the physical constitution may be taste sensitivity, the apparatus may be a cooking apparatus, and the decision may include deciding an apparatus control program for the cooking apparatus according to the taste sensitivity of the user.

According to this configuration, it is possible to cause the apparatus to execute appropriate control according to the taste sensitivity of the user.

In the control method, the physical constitution may be fatigue diathesis, the apparatus may be an information presentation device capable of presenting break information for encouraging a break, and the decision may include deciding one apparatus control program from among a plurality of apparatus control programs having different presentation timings of the break information according to the fatigue diathesis of the user.

According to the present configuration, it is possible to present appropriate break timing according to a fatigue diathesis of a user to the user.

In the control method, the determination may include detecting a single nucleotide polymorphism of the gene information, and determining the physical constitution on the basis of a detection result.

According to this configuration, since a physical constitution of a user is determined on the basis of a single nucleotide polymorphism of the gene information, the physical constitution of the user can be accurately determined.

An apparatus according to another aspect of the present disclosure includes: a reception part that receives control information for controlling execution of an apparatus control program decided on the basis of a physical constitution of a user determined from gene information of the user, and identification information of the user associated with the control information; a memory that stores the control information and the identification information in association with each other; a sensor for detecting a surrounding user; a specifying part that specifies identification information of the surrounding user from detection data of the sensor; and an execution part that specifies control information corresponding to the specified identification information from the memory and executes an apparatus control program corresponding to the specified control information.

According to this configuration, the control information for controlling the execution of the apparatus control program decided on the basis of a physical constitution of a user and the identification information of the user associated with the control information are stored in the memory in association with each other. Then, the identification information of a user around the apparatus is specified from detection data of the sensor, the control information corresponding to the specified identification information is specified from the memory, and the apparatus control program corresponding to the specified control information is executed. Therefore, the apparatus can execute control suitable for the surrounding user.

The present disclosure can be also implemented as a program for causing a computer to execute each characteristic function included in such a control method, or as a system that operates with the program. It is needless to say that such a computer program can be distributed using a computer-readable non-transitory recording medium such as a CD-ROM, or via a communication network such as the Internet.

Each of embodiments described below illustrates a specific example of the present disclosure. Numerical values, shapes, components, steps, an order of steps, and the like shown in the embodiments below are examples, and are not intended to limit the present disclosure. Further, among components in the embodiments below, a component that is not described in an independent claim representing a highest concept will be described as an optional component. In all the embodiments, respective contents can be combined.

### First Embodiment

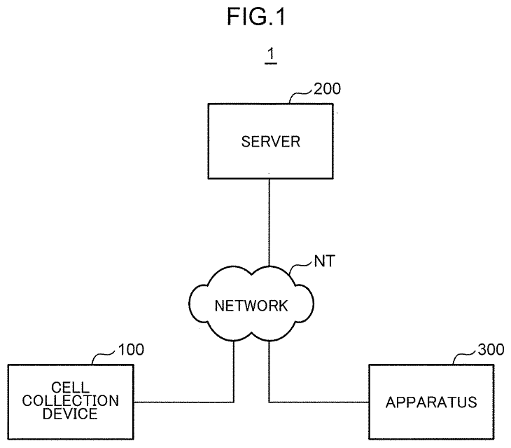

FIG. 1 is a diagram illustrating an example of an overall configuration of a control system 1 according to a first embodiment of the present disclosure. The control system 1 includes a cell collection device 100, a server 200, and an apparatus 300. The cell collection device 100, the server 200, and the apparatus 300 are communicably connected to each other via a network NT. The network NT is, for example, a public communication line such as the Internet. Note that the network NT may be a local area network. The cell collection device 100 and the apparatus 300 are installed, for example, in a house of the user. The cell collection device 100 and the apparatus 300 may be communicably connected via a home local network.

The cell collection device 100 is configured with, for example, a sequence decoding device also referred to as a DNA sequencer. The cell collection device 100 is a device that collects a cell of a user and extracts gene information of the user. The gene information includes information indicating a base sequence of DNA contained in the user's cell. The cell collection device 100 transmits the extracted gene information to the server 200 in association with a user ID that is identification information of the user.

The server 200 is configured with a cloud server including one or more computers, for example. The server 200 determines a physical constitution of a user on the basis of the gene information transmitted from the cell collection device 100, decides an apparatus control program suitable for the physical constitution, and transmits the decided apparatus control program to the apparatus 300 in association with the user ID.

The apparatus 300 is configured with an electric apparatus. The electric apparatus is, for example, a household electric apparatus such as an air conditioning apparatus, a cooking apparatus, a television, or a personal computer. The apparatus 300 executes the apparatus control program transmitted from the server 200.

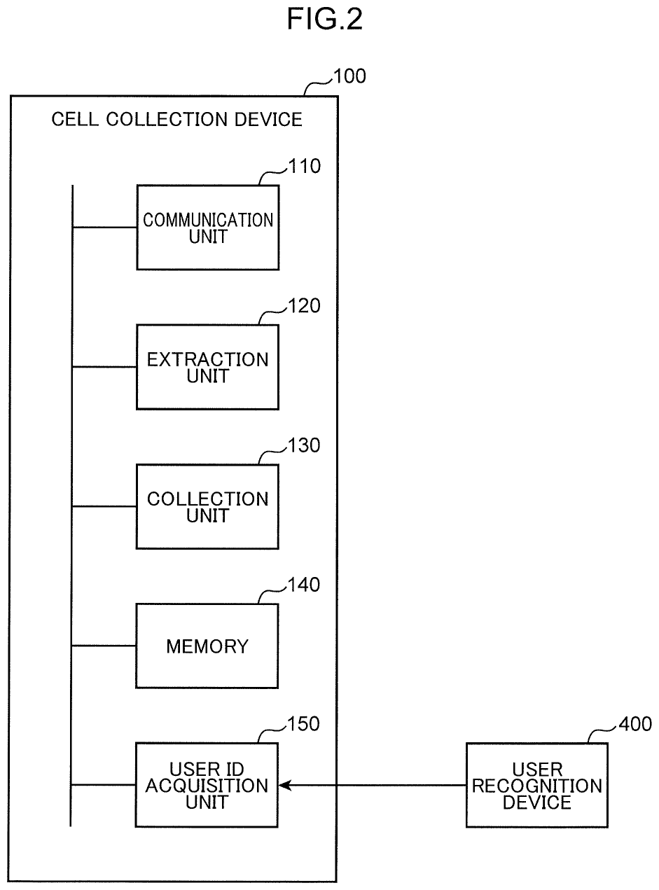

The foregoing is the entire configuration of the control system 1. Next, details of each component of the control system 1 will be described. FIG. 2 is a block diagram illustrating an example of a configuration of the cell collection device 100. The cell collection device 100 includes a communication unit 110, an extraction unit 120, a collection unit 130, a memory 140, and a user ID acquisition unit 150.

The communication unit 110 is configured with a communication circuit that connects the cell collection device 100 to the network NT. The communication unit 110 transmits gene information extracted by the extraction unit 120 to the server 200 in association with a user ID of a user who has provided the gene information.

The collection unit 130 collects a cell of a user and supplies the collected cell to the extraction unit 120.

The extraction unit 120 includes a labeling part, a light source, an image sensor, and a processor. The labeling part applies a fluorescent label for each type of base (A, T, G, C) to DNA of a cell collected by the collection unit 130. The light source irradiates DNA having an applied fluorescent label with light. The image sensor detects fluorescence emitted from DNA by light from the light source. The processor generates gene information including information indicating a base sequence of DNA on the basis of fluorescence detected by the image sensor.

The memory 140 is configured with a nonvolatile storage device that cannot be rewritten such as a flash memory. The memory 140 stores, for example, a user ID.

The user ID acquisition unit 150 includes, for example, a communication interface for communicating with a user recognition device 400, and acquires a user ID of a user who has provided gene information. Alternatively, the user ID acquisition unit 150 may acquire a user ID from memory 140. Alternatively, the user ID acquisition unit 150 may acquire a user ID input by a user using an operation device (not illustrated).

For example, when the cell collection device 100 is mounted on an electric apparatus (e.g., an electric toothbrush) used for each individual user, the memory 140 can store an user ID of a user who provides the gene information. In this case, the user ID acquisition unit 150 may acquire the user ID stored in the memory 140 as a user ID of a user who provides the gene information.

For example, the cell collection device 100 may be mounted on an operation switch of a lighting apparatus or the like which a user frequently touches. In this case, the cell collection device 100 may acquire the user ID from the user recognition device 400 provided around the operation switch.

When detecting a user touching the operation switch, the user recognition device 400 transmits a user ID of the user who has touched the operation switch to the user ID acquisition unit 150. The user recognition device 400 may detect the user who has touched the operation switch using, for example, image recognition processing. Specifically, the user recognition device 400 includes a camera, a communication unit, and a processor. The camera constantly captures an image around the operation switch. The processor executes image processing on image data captured by the camera, and detects whether or not a certain user has touched the operation switch. In a case where the processor detects a certain user touching the operation switch, the processor executes face recognition processing to determine to which user among users registered in advance, the user corresponds, and detects a user ID of the determined user as the user ID of the user who has touched the operation switch. The communication unit may input the user ID detected by the processor to the user ID acquisition unit 150.

Alternatively, the user recognition device 400 may detect the user ID by fingerprint recognition. In this case, the user recognition device 400 is configured with a fingerprint recognition device provided in the operation switch.

Alternatively, the user recognition device 400 may be mounted on the apparatus 300. In this case, the user recognition device 400 may detect a user from which a cell is collected by the cell collection device 100 in response to a user ID acquisition request output from the user ID acquisition unit 150, and input a user ID of the user to the user ID acquisition unit 150.

In a case where the cell collection device 100 is mounted on an electric toothbrush, the collection unit 130 may collect saliva of the user and collect a cell from the collected saliva. In a case where the cell collection device 100 is mounted on the operation switch, the collection unit 130 may collect sweat of a user and collect a cell of the user from the collected sweat.

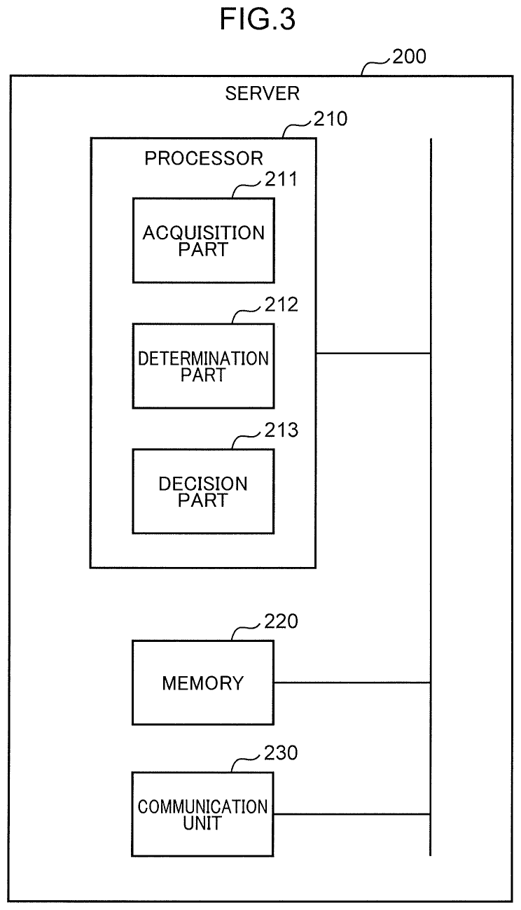

FIG. 3 is a block diagram illustrating an example of a configuration of the server 200. The server 200 includes a processor 210, a memory 220, and a communication unit 230 (an example of a transmission part). The processor 210 is configured with, for example, a CPU. The processor 210 includes an acquisition part 211, a determination part 212, and a decision part 213. The acquisition part 211 to the decision part 213 may be implemented by execution of a predetermined program by the processor 210, or may be configured with a dedicated hardware circuit.

The acquisition part 211 acquires a user ID and the gene information transmitted by the cell collection device 100 via the communication unit 230. The acquisition part 211 applies a time stamp to the acquired gene information, and stores the gene information to which the time stamp is applied in the memory 220 in association with the user ID. As a result, time-series data of the gene information for each user is accumulated in the memory 220.

The determination part 212 determines a physical constitution of a user who has provided the gene information on the basis of the gene information acquired by the acquisition part 211. Here, the determination part 212 detects a single nucleotide polymorphism (SNP) of a base sequence indicated by the gene information and a type of SNP, and determines a physical constitution of the user on the basis of a detection result. Specifically, the determination part 212 determines whether or not a SNP has appeared at a predetermined gene locus on a base sequence related to a physical constitution to be determined. In a case where a SNP has appeared at the predetermined gene locus, the determination part 212 specifies a type of the SNP. Then, the determination part 212 determines a physical constitution to be determined from the specified type. For example, the determination part 212 may determine a SNP type from a pattern of bases of SNP located at the same gene locus in homologous chromosomes.

Although base sequences of a human being are 99.9% identical, 0.1% are different. This difference causes a difference in appearance, ability, physical constitution, and the like. When a difference in base sequence appears at a frequency of 1% or more in a certain human group, the difference in the base sequence is called polymorphism. When a difference in base sequence appears at a frequency of 1% or less, the difference in base sequence is called mutation or rare variation. Polymorphism has various types, and among them, SNP is one in which one base is replaced by another base. SNP is estimated to be present at a probability of one in 500 to 1000 bases, and is estimated to be present at about ten million locations.

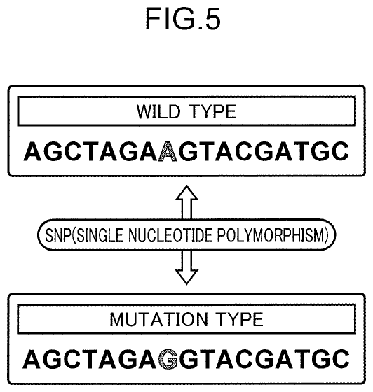

FIG. 5 is an explanatory diagram of SNP. In the example of FIG. 5, A in a normal gene sequence (wild type) is mutated to G.

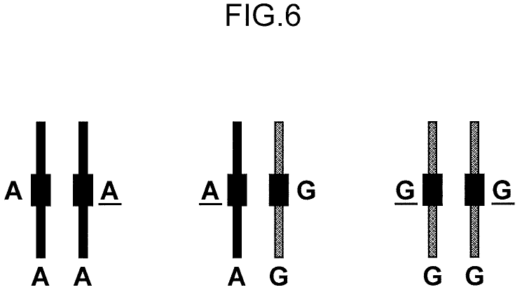

A humans being inherits one gene sequence from each of his/her father and mother, and there are three combinations. Therefore, one SNP has three types. FIG. 6 is an explanatory diagram of SNP types. For example, in the SNP mutation of A to G as shown in the example of FIG. 5, there are three SNP types, AA, AG, and GG.

For example, when SNPs of parents of a child are of the AG type, the child will inherit one A sequence and one G sequence of each of the parents. Therefore, the SNP mutation of A to G will have three types, AA, AG, and GG as shown in FIG. 6.

While there are many SNPs, it has been shown that a specific SNP is associated with a specific disease. Such SNP associated with a certain disease is referred to as “disease-related SNP.”

Examples of a disease-related SNP include a “metabolically related SNP”. “Metabolically-related SNP” is a SNP related to “metabolic syndrome”. A type of “metabolically related SNP” makes it possible to determine whether a person in question has a “physical constitution for getting fat” or a “physical constitution not for getting fat”.

Other examples of the disease-related SNP includes a SNP related to an alcoholysis enzyme. A combination of GG as a SNP type related to an alcoholysis enzyme is called GG homotype. A person having this SNP type is resistant to alcohol. A combination of AG as a SNP type related to an alcoholysis enzyme is called AG heterozygous type. A person having this SNP type is naturally vulnerable to alcohol. A combination of AA as a SNP type related to an alcoholysis enzyme is called AA homotype. A person having this SNP type lacks metabolic activity for alcohol and cannot drink alcohol by nature.

As described above, when a specific SNP and a type of the SNP are known for a certain person, a physical constitution of the person can be found.

Among physical constitutions known from a base sequence include an onset risk of a disease. Examples of diseases include type 2 diabetes, myocardial infarction, cerebral infarction, cluster headache, LDL-cholesterol, insomnia, atopic dermatitis, duodenal ulcer, and nicotine dependence.

Further, physical constitutions known from a base sequence include a tendency of human characteristics. Examples of characteristic tendencies include a fatigue tendency, a life span tendency, a tendency of a height, a tendency of a hair thickness, a tendency of a color of an eye, a tendency of an information processing speed, a tendency of a calculation speed, and a tendency of a memory.

Further, physical constitutions known from a base sequence include vitamin concentration, development tendency of fast and slow muscles, taste sensitivity, and the like.

By thus analyzing a base sequence, it is possible to determine various physical constitutions. Therefore, the determination part 212 can determine these physical constitutions from SNP and a type of SNP. In the present embodiment, the determination part 212 determines thermal diathesis, taste sensitivity, and fatigue diathesis in particular. Thermal diathesis represents a physical constitution such as sensitive to heat or sensitive to cold. Taste sensitivity represents sensitivities to taste, and among them, for example, sweetness sensitivity represents a physical constitution having high sensitivity to sweetness or low sensitivity to sweetness. In the following, as taste sensitivity, sweetness sensitivity will be described as an example. Fatigue diathesis is a physical constitution such as being easily fatigued or being less easily fatigued. For example, the determination part 212 may determine a physical constitution of a user by referring to information defining in advance a correspondence relationship between a type of SNP to be determined and a physical constitution.

The decision part 213 decides one apparatus control program from among a plurality of apparatus control programs for controlling the apparatus 300 on the basis on the physical constitution determined by the determination part 212. The decision part 213 transmits the determined one apparatus control program to the apparatus 300 via the communication unit 230 in association with the user ID.

For example, in a case where the apparatus 300 is an air conditioning apparatus, there are provided, as an apparatus control program for the air conditioning apparatus, apparatus control programs for standard, heat-sensitive, and cold-sensitive users.

The apparatus control program for a heat-sensitive user is, for example, an apparatus control program for operating an air conditioning apparatus at a set temperature lower than a standard set temperature. The apparatus control program for a standard user is, for example, an apparatus control program for operating an air conditioning apparatus at the standard set temperature. The cold apparatus control program for a cold-sensitive user is, for example, an apparatus control program for operating an air conditioning apparatus at a set temperature higher than the standard set temperature.

When the determination part 212 determines that the user has a heat-sensitive thermal diathesis, the decision part 213 selects the apparatus control program for a heat-sensitive user. When the determination part 212 determines that the user has a cold-sensitive thermal diathesis, the decision part 213 selects the apparatus control program for a cold-sensitive user. When the determination part 212 determines that the user has a standard thermal diathesis, the decision part 213 selects the apparatus control program for a standard user.

For example, in a case where the apparatus 300 is a cooking apparatus, as apparatus control programs for a cooking apparatus, there are provided an apparatus control program for a user having high sweetness sensitivity, an apparatus control program for a user having standard sweetness sensitivity, and an apparatus control program for a user having low sweetness sensitivity.

The apparatus control program for a user having high sweetness sensitivity is, for example, an apparatus control program for operating a cooking apparatus with a recipe using an amount of sugar suppressed to be smaller than usual, or an apparatus control program for suggesting the recipe to the user. The apparatus control program for a user having low sweetness sensitivity is, for example, an apparatus control program for operating a cooking apparatus with a recipe using an amount of sugar suppressed to be larger than usual, or an apparatus control program for suggesting the recipe to the user. The apparatus control program for a user having standard sweetness sensitivity is, for example, an apparatus control program for operating a cooking apparatus with a recipe using an usual amount of sugar, or an apparatus control program for suggesting the recipe to the user.

When the determination part 212 determines that the user has high sweetness sensitivity, the decision part 213 selects the apparatus control program for a user having high sweetness sensitivity. When the determination part 212 determines that the user has low sweetness sensitivity, the decision part 213 selects the apparatus control program for a user having low sweetness sensitivity. When the determination part 212 determines that the user has standard sweetness sensitivity, the decision part 213 selects the apparatus control program for a user having standard sweetness sensitivity.

For example, in a case where the apparatus 300 is an information presentation device capable of presenting break information for urging a user to take a break, the apparatus control program includes, for example, an apparatus control program for a user having a fatigue diathesis of being easily fatigued, an apparatus control program for a user having a normal fatigue diathesis, and an apparatus control program for a user having a fatigue diathesis of being less easily fatigued.

The apparatus control program for a user having a fatigue diathesis of being easily fatigued is, for example, an apparatus control program having presentation timing for the break information that is earlier than normal presentation timing by a predetermined time period. The apparatus control program for a user having a normal fatigue diathesis is, for example, an apparatus control program having normal presentation timing for the break information. The apparatus control program for a user having a fatigue diathesis of being less easily fatigued is, for example, an apparatus control program having presentation timing for the break information that is later than the standard presentation timing by a predetermined time. The information presentation device periodically presents the break information after a power source is turned on, for example. Therefore, the apparatus control program changes a cycle of presenting the break information according to a physical constitution of a user.

When the determination part 212 determines that the user has a fatigue diathesis of being easily fatigued, the decision part 213 selects the apparatus control program for a user having a fatigue diathesis of being easily fatigued. When the determination part 212 determines that the user has a normal fatigue diathesis, the decision part 213 selects the apparatus control program for a user who has a normal fatigue diathesis. When the determination part 212 determines that the user has a fatigue diathesis of being less easily fatigued, the decision part 213 selects the apparatus control program for a user having a fatigue diathesis of being less easily fatigued.

The memory 220 is configured with a storage device such as a hard disk drive or a solid state drive. The memory 220 stores a plurality of apparatus control programs for each type of the apparatus 300. Here, the memory 220 stores the apparatus control program and the physical constitution information indicating a physical constitution in association with each other. For example, an apparatus control program for an air conditioning apparatus will be described as an example. The memory 220 stores the apparatus control programs respectively for heat-sensitive, cold-sensitive, and standard users in association with physical constitution information for cold-sensitive, heat-sensitive, and standard users, respectively.

The communication unit 230 is configured with a communication circuit that connects the server 200 to the network NT. The communication unit 230 transmits an apparatus control program decided by the decision part 213 and decided user ID of a user corresponding to the decided apparatus control program to the apparatus 300 in association with each other.

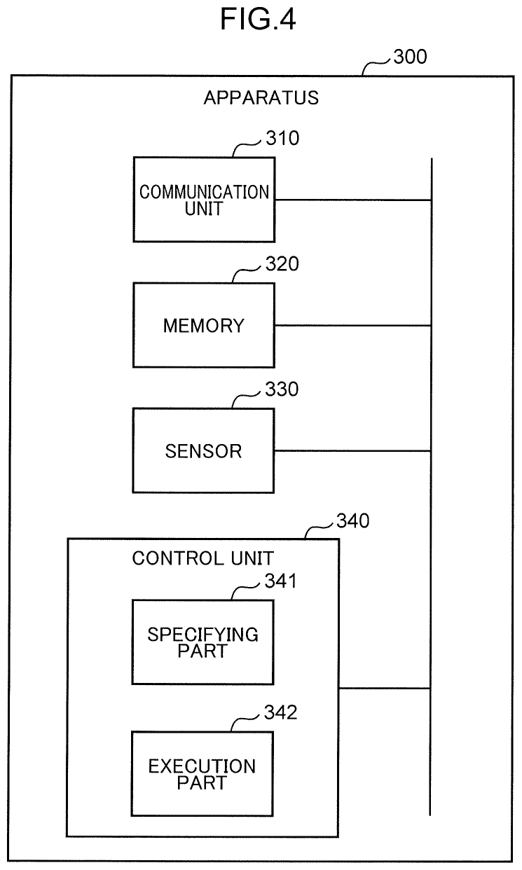

FIG. 4 is a block diagram illustrating an example of a configuration of the apparatus 300. The apparatus 300 includes a communication unit 310 (an example of a reception part), a memory 320, a sensor 330, and a control unit 340. The communication unit 310 is configured with a communication circuit that connects the apparatus 300 to the internal network NT. The communication unit 310 receives an apparatus control program and a user ID transmitted from the server 200.

The memory 320 is configured with a nonvolatile storage device that can be rewritten such as a flash memory. The memory 320 stores an apparatus control program and a user ID received by the communication unit 310 from the server 200 in association with each other. Further, the memory 320 stores a feature value of one or more users registered in advance and an user ID in association with each other. A feature value of a user is, for example, a feature value of a face, a feature value of a voice, or the like.

The sensor 330 is a sensor for detecting a surrounding user. The sensor 330 differs depending on the apparatus 300. For example, when the apparatus 300 is an air conditioning apparatus or an information presentation device, the sensor 330 is configured with an image sensor. For example, in a case where the apparatus 300 is a cooking apparatus, the sensor 330 is configured with a microphone.

Here, the information presentation device is a device capable of presenting some information to a user, such as a television, a personal computer, a tablet terminal, a smartphone, or a smart speaker. Note that in a case where the apparatus 300 is configured with a smart speaker, the sensor 330 may be configured with a microphone.

The control unit 340 is configured with a processor such as a CPU. The control unit 340 includes a specifying part 341 and an execution part 342. The specifying part 341 detects a user from detection data of the sensor 330 and specifies a user ID of the detected user.

For example, in a case where the sensor 330 is an image sensor, the specifying part 341 detects a user by executing predetermined image recognition processing on image data that is detection data. Then, the specifying part 341 may sequentially compare a feature value of a face registered in the memory 320 with respect to a face image included in the image data, and specify an user ID corresponding to a feature value whose similarity is equal to or greater than a threshold and which is the maximum as a user ID of a user included in the image data.

For example, in a case where the sensor 330 is a microphone, the specifying part 341 detects voice of a user by executing predetermined voice recognition processing on sound data that is detection data. Then, the specifying part 341 may sequentially compare the sound data with a feature value of voice of one or more users registered in advance to specify a user ID.

Although the processing of specifying a user ID using a feature value registered in advance in the memory 320 has been described here, this is just an example. For example, the specifying part 341 may specify a user ID by inputting detection data of the sensor 330 to a user specifying model generated in advance through machine learning such as a neural network.

The execution part 342 acquires an apparatus control program corresponding to a user ID specified by the specifying part 341 from the memory 320 and executes the apparatus control program. As a result, control of the apparatus 300 suitable for a physical constitution of a user around the apparatus 300 is realized.

Note that in a case where a plurality of users is included in the detection data of the sensor 330, the specifying part 341 may specify a user ID of a user who has operated the apparatus 300 most recently. For example, in a case of an air conditioning apparatus, an user ID of a user who has most recently turned on a power source of the air conditioning apparatus is specified. Alternatively, the specifying part 341 may determine a user who mainly operates the apparatus 300 according to attributes (e.g., father, mother, child, and the like) of a plurality of users and specify a user ID of the user. For example, in a case of a cooking apparatus, a user ID of a user (e.g., mother) who mainly cooks is specified.

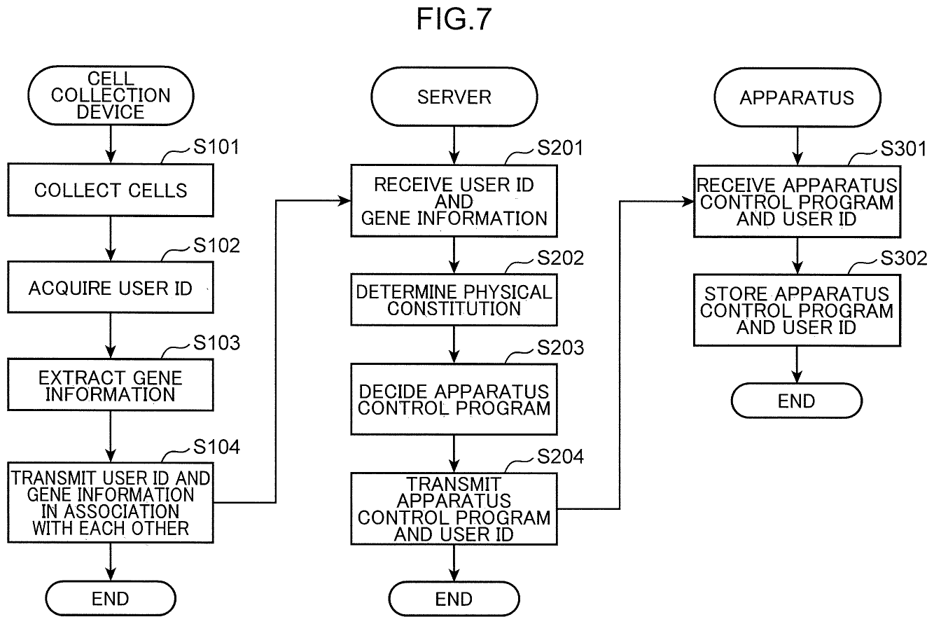

FIG. 7 is a flowchart illustrating an example of processing executed when the control system 1 according to the first embodiment of the present disclosure decides an apparatus control program. Note that in this flowchart, for convenience of description, processing for deciding one apparatus control program for a certain one apparatus 300 in a certain one house will be described as an example.

In Step S101, the collection unit 130 of the cell collection device 100 collects cells of a user. In Step S102, the user ID acquisition unit 150 acquires a user ID of the user whose cells have been collected. For example, as described above, the user ID acquisition unit 150 may acquire a user ID stored in advance in the memory 140, or may acquire a user ID from the user recognition device 400, or may acquire a user ID input by the user.

In Step S103, the extraction unit 120 extracts gene information from the collected cells. In Step S104, the communication unit 110 transmits the user ID acquired in Step S102 and the gene information extracted in Step S103 to the server 200 in association with each other.

In Step S201, the communication unit 230 of the server 200 receives the user ID and the gene information. In Step S202, the determination part 212 determines a physical constitution of the user who has provided his/her gene information from the gene information received in Step S201. For example, the determination part 212 may specify a user's physical constitution from SNP and a type of the SNP as described above.

In Step S203, the decision part 213 decides one apparatus control program corresponding to the physical constitution determined in Step S202 from a plurality of apparatus control programs predetermined for the apparatus 300. For example, as described above, when a user has a thermal diathesis of being sensitive to heat and the apparatus 300 to be controlled is an air conditioning apparatus, an apparatus control program for a heat-sensitive user is decided.

In Step S204, the communication unit 230 transmits the apparatus control program decided in Step S203 and the user ID received in Step S201 to the apparatus 300 in association with each other.

In Step S301, the communication unit 310 of the apparatus 300 receives the apparatus control program and the user ID.

In Step S302, the communication unit 310 stores the apparatus control program and the user ID received in Step S301 in the memory 320 in association with each other.

As described above, the apparatus 300 can acquire an apparatus control program according to a physical constitution of an individual user in a house.

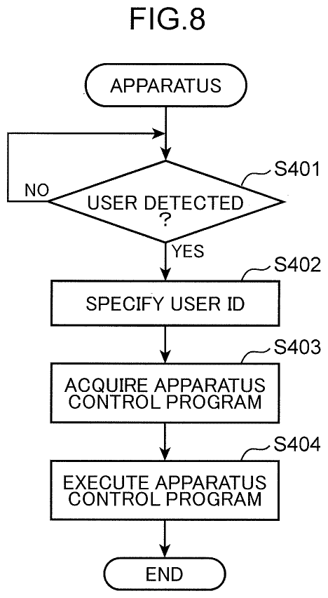

FIG. 8 is a flowchart illustrating an example of processing executed when the apparatus 300 executes the apparatus control program. In Step S401, the specifying part 341 detects a user from detection data of the sensor 330. When a user is detected (YES in Step S401), the processing proceeds to Step S402, and when no user is detected (NO in Step S402), the processing returns to Step S401.

In Step S402, the specifying part 341 specifies a user ID of the user detected in Step S401. In Step S403, the execution part 342 acquires an apparatus control program corresponding to the user ID specified in Step S402 from the memory 320.

In Step S404, the execution part 342 executes the apparatus control program acquired in Step S403.

As described above, according to the present embodiment, since a physical constitution of a user is determined on the basis of gene information, the physical constitution of the user can be accurately determined. One apparatus control program is decided from the plurality of apparatus control programs on the basis of the determined physical constitution, and the decided one apparatus control program and the identification information are transmitted to the apparatus 300. Therefore, the apparatus 300 can specify an apparatus control program corresponding to a user ID. As a result, the apparatus 300 can execute control suitable for an individual user.

### Second Embodiment

The control system 1 of a second embodiment detects an expression of a predetermined gene on the basis of a temporal change in gene information. In the present embodiment, the same components as those in the first embodiment are denoted by the same reference numerals, and description thereof will be omitted.

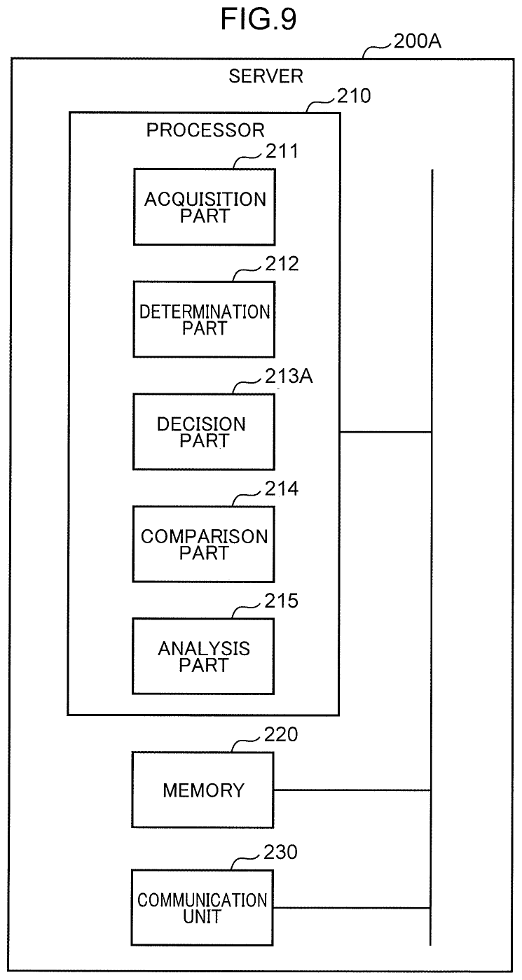

FIG. 9 is a block diagram illustrating an example of a configuration of a server 200A according to the second embodiment of the present disclosure. The second embodiment is different from the first embodiment in that the processor 210 further includes a comparison part 214 and an analysis part 215. Function of a decision part 213A is changed from that of the decision part 213 of the first embodiment.

The comparison part 214 compares a plurality of pieces of gene information of a certain user accumulated in the memory 220, and detects whether or not there occurs a change in the gene information. For example, the comparison part 214 may compare the most recent gene information with the second recent gene information.

When a change in gene information is detected by the comparison part 214, the analysis part 215 detects presence or absence of an expression of a predetermined gene by analyzing the changed gene information.

A molecular structure of a gene is affected by “environment” and “breeding”. In other words, an expression of a certain gene depends on a living environment and a lifestyle. Many acquired phenomena without a change in a gene sequence, such as aging or canceration, are related to gene expression. A gene is stored in a nucleus of a cell in a state of winding around a protein called histone and being further folded. A structure in which DNA is wound around histones is called “chromatin”. One chromatin contains approximately 150 base pairs of DNA.

Epigenetics studies have revealed that gene expression is related to a shape of chromatin. In a state where a chromatin structure is hardened, a gene switch for expressing a gene is turned off, and conversely in a state where the chromatin structure is opened, the gene switch is turned on. Furthermore, this structural change in chromatin is caused by chemical modification of histone proteins and DNA wound around them. For example, when histone proteins are methylated, chromatin becomes clumped to turn off a gene switch. When histone proteins are acetylated, chromatin becomes open to turn on the gene switch. Also for DNA wound around the histone proteins, the gene switch is turned off by methylation of C (cytosine) in the DNA.

Therefore, the analysis part 215 estimates an expression of a gene by detecting a change in a base sequence of a predetermined gene locus related to a certain gene. Specifically, when a change in a base sequence is detected by the comparison part 214, the analysis part 215 determines whether or not the changed gene locus is a gene locus related to a target gene. Then, in a case where the changed gene locus is a gene locus related to the target gene, the analysis part 215 may determine that the gene might have been expressed. Furthermore, in a case where an expression of a gene is detected, the analysis part 215 may determine that a physical constitution of a user related to the gene has changed. For example, in a case where an expressed gene is a gene indicating a physical constitution change from sensitive to heat to sensitive to cold, the analysis part 215 may determine that the user's thermal diathesis has changed from sensitive to heat to sensitive to cold. For example, in a case where the expressed gene is a gene indicating a change from a physical constitution having high sweetness sensitivity to a physical constitution having low sweetness sensitivity, the analysis part 215 may determine that the user's physical constitution has changed from the high sweetness sensitivity to the low sweetness sensitivity. For example, in a case where the expressed gene is a gene indicating a change from a physical constitution of being easily fatigued to a physical constitution of being less easily fatigued, the analysis part 215 may determine that the fatigue diathesis of the user has changed from a physical constitution of being easily fatigued to a physical constitution of being less easily fatigued.

In a case where the expression of the gene is detected by the analysis part 215, the decision part 213A decides an apparatus control program on the basis of a detection result. Specifically, when the analysis part 215 determines that the physical constitution of the user has changed as the gene is expressed, the decision part 213A decides an apparatus control program according to the changed physical constitution. The decision part 213A then transmits the decided apparatus control program to the apparatus 300 via the communication unit 230 in association with the user ID.

Upon receiving the apparatus control program, the apparatus 300 updates an apparatus control program for the corresponding user stored in the memory 320 with the received apparatus control program. As a result, the apparatus 300 can execute an apparatus control program suitable for the changed physical constitution.

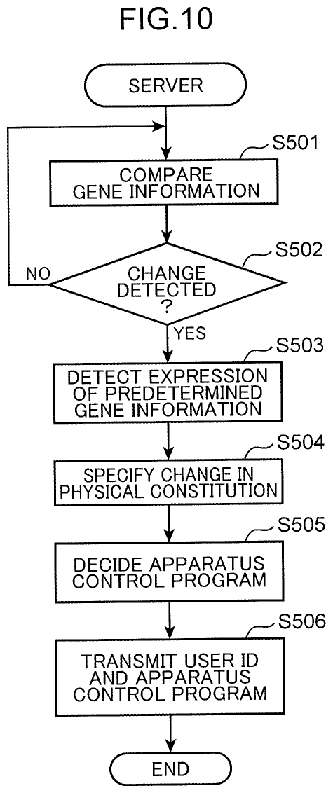

FIG. 10 is a flowchart illustrating an example of processing of the server 200 according to the second embodiment. Note that the flowchart illustrated in FIG. 10 may be executed each time the server 200 receives new gene information, or may be executed each time a predetermined number of pieces of new gene information is accumulated.

In Step S501, the comparison part 214 compares the gene information of a certain user stored in the memory, and detects presence or absence of a change in a base sequence. When the change of the gene information is detected in Step S502 (YES in Step S502), the processing proceeds to Step S503, and when no change of the gene information is detected (NO in Step S502), the processing returns to Step S501.

In Step S503, the analysis part 215 detects an expression of a predetermined gene. What are detected here are expressions of genes related to thermal diathesis, sweetness sensitivity, and fatigue diathesis. This is just an example, and what are detected may be expressions of genes related to various diseases exemplified in the first embodiment, or may be expressions of genes related to a tendency of human characteristics.

In Step S504, the analysis part 215 specifies a change in the physical constitution of the user from the expression of the gene detected in Step S503. In Step S505, the decision part 213A decides an apparatus control program corresponding to the changed physical constitution.

In Step S506, the decision part 213A transmits the decided apparatus control program to the apparatus 300 in association with the user ID.

As described above, according to the present embodiment, since an expression of a predetermined gene is detected and an apparatus control program is decided on the basis of a detection result, it is possible to decide an appropriate apparatus control program in consideration of a change in a physical constitution of a user.

### Third Embodiment

In the first and second embodiments, the server 200 transmits the apparatus control program to the apparatus 300 in association with the user ID. In the third embodiment, the server 200 transmits control information for executing an apparatus control program to the apparatus 300. Note that in the present embodiment, the same components as those in the first embodiment are denoted by the same reference numerals, and description thereof will be omitted.

Refer to FIG. 3. In the present embodiment, the decision part 213 of the server 200 decides one apparatus control program from among a plurality of apparatus control programs on the basis of a physical constitution determined by the determination part 212, and transmits control information for executing the decided one apparatus control program to the apparatus 300 via the communication unit 230 in association with the user ID. The control information includes, for example, physical constitution information indicating a physical constitution of a user determined by the determination part 212.

Refer to FIG. 4. In the present embodiment, the memory 320 of the apparatus 300 stores a plurality of presentation control programs associated with physical constitution information in advance. The memory 320 further stores the control information transmitted from the server 200 and a user ID in association with each other.

The execution part 342 acquires an apparatus control program associated with physical constitution information corresponding to a user ID specified by the specifying part 341 from the memory 320 and executes the acquired apparatus control program.

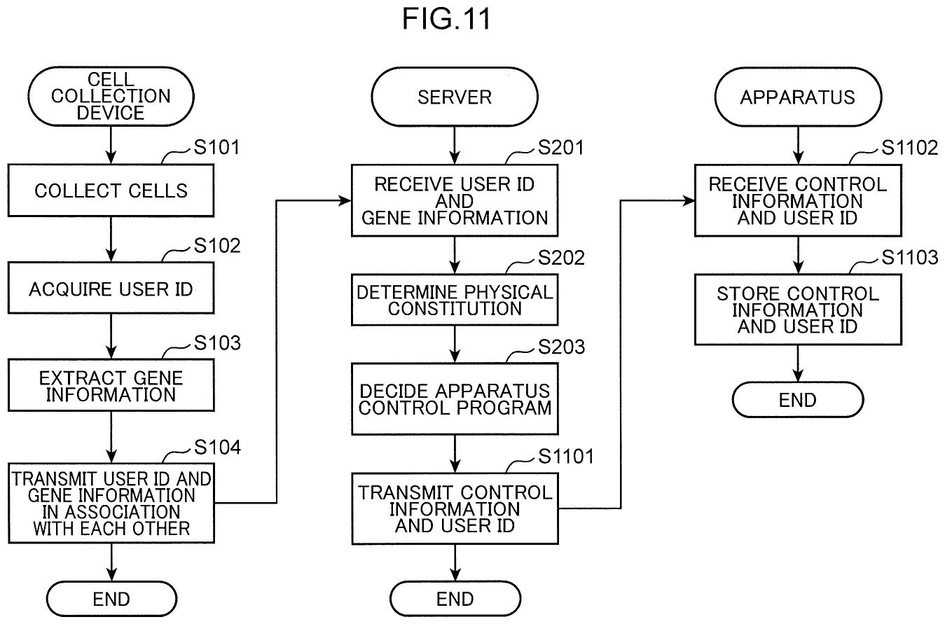

FIG. 11 is a flowchart illustrating an example of processing of the control system 1 according to the third embodiment of the present disclosure. In the present flowchart, the same processing as that in FIG. 7 is denoted by the same processing numeral, and description thereof will be omitted.

In Step S1101 subsequent to Step S203, the communication unit 230 transmits the control information for executing the apparatus control program decided in Step S203 and the user ID to the apparatus 300.

In Step S1102, the communication unit 310 of the apparatus 300 receives the user ID and the control information. In Step S1103, the communication unit 310 stores the received user ID and control information in the memory 320 in association with each other.

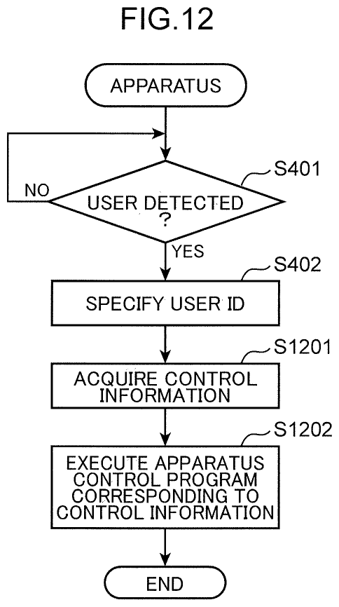

FIG. 12 is a flowchart illustrating an example of processing executed when the apparatus 300 executes the apparatus control program in the third embodiment of the present disclosure. In FIG. 12, the same processing as that in FIG. 8 is denoted by the same processing numeral, and description thereof will be omitted.

In Step S1201 subsequent to Step S402, the execution part 342 acquires control information corresponding to the user ID specified by the specifying part 341 from the memory 320.

In Step S1202, the execution part 342 acquires an apparatus control program corresponding to physical constitution information included in the control information from the memory 320 and executes the acquired apparatus control program.

As described above, according to the third embodiment, since the plurality of apparatus control programs is stored in advance in the apparatus 300 in association with the physical constitution information, the server 200 can cause the apparatus 300 to execute an apparatus control program suitable for a physical constitution of a user without transmitting the apparatus control program to the apparatus 300.

### Modification

Modifications set forth below can be adopted in the present disclosure.

(1) The third embodiment is also applicable to the second embodiment. Specifically, while in the second embodiment, when a change in a physical constitution is specified, an apparatus control program corresponding to the changed physical constitution is transmitted, control information including physical constitution information indicating the changed physical constitution may be transmitted instead of the transmission of the apparatus control program.

(2) Although in the first to third embodiments, the server 200 acquires the gene information from the cell collection device 100 installed in a house of a user, the present disclosure is not limited thereto. For example, the server 200 may acquire gene information of the user measured by an external organization together with an user ID of the user.

(3) In the present disclosure, the method for determining SNP and a SNP type is not limited to the above-described method, and for example, it is possible to employ a restriction fragment length polymorphism (RFLP) method, a single strand conformation polymorphism (SSCP) method, an SSCP method, a TaqMan PCR method, an SNaP Shot method, an Invader method, a mass spectrometry method, or a method using a DNA microarray. When these methods are adopted, the gene information may include information indicating SNP and a SNP type.

For example, in a case where a method using a DNA macroarray is adopted, the cell collection device 100 is configured with a DNA microarray. In this case, the cell collection device 100 may transmit information on a specific SNP and a type of the SNP out of collected cells to the server 200 as gene information. The determination part 212 of the server 200 may determine a physical constitution of the user from the SNP and the SNP type included in the gene information.

(4) Although the cell collection device 100, the server 200, and the apparatus 300 are separate devices, the present disclosure is not limited thereto, and they may be configured by one device. In this case, “transmit the acquired identification information and control information for causing the apparatus to execute the decided apparatus control program in association with each other” means that the identification information and the control information are transmitted in association with each other in the one device.

(5) Although in the first to third embodiments, it has been described that one apparatus control program is decided from among a plurality of apparatus control programs, the present disclosure is not limited thereto.

### INDUSTRIAL APPLICABILITY

According to the present disclosure, since it is possible to execute control suitable for a physical constitution of a user, it is useful for realizing comfortable control for the user.

1. A control method of an apparatus, the method comprising: by a computer
acquiring identification information for specifying a user and gene information of the user associated with the identification information,
determining a physical constitution of the user on the basis of the acquired gene information,
deciding an apparatus control program for controlling the apparatus on the basis of the determined physical constitution, and
transmitting, in association with each other, the acquired identification information and control information for causing the apparatus to execute the decided apparatus control program.

2. The control method according to claim 1, wherein the gene information includes information indicating a base sequence of a nucleic acid of the user.

3. The control method according to claim 1, further comprising detecting an expression of a predetermined gene on the basis of a temporal change in the acquired gene infoimation, and deciding the apparatus control program on the basis of a detection result.

4. The control method according to claim 1, wherein the physical constitution is at least one of thermal diathesis, taste sensitivity, and fatigue diathesis.

5. The control method according to claim 1, wherein the control information includes the apparatus control program.

6. The control method according to claim 1, wherein the control information includes information for causing the apparatus having a plurality of apparatus control programs to execute the decided apparatus control program.

7. The control method according to claim 3, wherein
the physical constitution is thermal diathesis,
the apparatus is an air conditioning apparatus, and
the decision includes deciding an apparatus control program for the air conditioning apparatus according to the thermal diathesis of the user.

8. The control method according to claim 3, wherein
the physical constitution is taste sensitivity,
the apparatus is a cooking apparatus, and
the decision includes deciding an apparatus control program for the cooking apparatus according to the taste sensitivity of the user.

9. The control method according to claim 3, wherein
the physical constitution is fatigue diathesis,
the apparatus is an information presentation device capable of presenting break information for encouraging a break, and
the decision includes deciding one apparatus control program from among a plurality of apparatus control programs having different presentation timings of the break information according to the fatigue diathesis of the user.

10. The control method according to claim 1, wherein the determination includes detecting a single nucleotide polymorphism of the gene information, and determining the physical constitution on the basis of a detection result.

11. A control device of an apparatus, comprising:
an acquisition part that acquires identification information for specifying a user and gene information of the user associated with the identification information;
a determination part that determines a physical constitution of the user on the basis of the acquired gene information;
a decision part that decides an apparatus control program for controlling the apparatus on the basis of the determined physical constitution; and
a transmission part that transmits the acquired identification information and control information for causing the apparatus to execute the decided apparatus control program in association with each other.

12. A non-transitory computer-readable recording medium storing a program for causing a computer to execute a control method of an apparatus, the program causing the computer to:
acquiring identification information for specifying a user and gene information of the user associated with the identification information;
determining a physical constitution of the user on the basis of the acquired gene information;
deciding an apparatus control program for controlling the apparatus on the basis of the determined physical constitution; and
transmitting the acquired identification information and control information for causing the apparatus to execute the decided apparatus control program in association with each other.

13. An apparatus comprising:
a reception part that receives control information for controlling execution of an apparatus control program decided on the basis of a physical constitution of a user determined from gene information of the user, and identification information of the user associated with the control information;
a memory that stores the control information and the identification information in association with each other;
a sensor for detecting a surrounding user;
a specifying part that specifies identification information of the surrounding user from detection data of the sensor; and
an execution part that specifies control information corresponding to the specified identification information from the memory and executes an apparatus control program corresponding to the specified control information.
